(19) United States
(12) Patent Application Publication
(10) Pub. No.: US 2017/0234847 A1
(43) Pub. Date: Aug. 17, 2017

(54) Lanthanide-Doped Nanoparticle Compositions for Detecting Microorganisms

(71) Applicant: BacTrac Technologies LLC, Apple Valley, MN (US)

(72) Inventors: Vivian Hsiao, Roseville, MN (US); Vincent Mar, Apple Valley, MN (US)

(21) Appl. No.: 15/431,530

(22) Filed: Feb. 13, 2017

Related U.S. Application Data
(60) Provisional application No. 62/295,053, filed on Feb. 13, 2016.

(51) Int. Cl. G01N 33/12; C12Q 1/10; C09K 11/77

(52) U.S. Cl. CPC G01N 33/12; C09K 11/7777; C12Q 1/10

(57) ABSTRACT

A particulate lanthanide-doped material comprising an inorganic host phosphor and a lanthanide ion dopant. With the lanthanide-doped material being in an oxidized state, photoluminescence is suppressed. Photoluminescence of the material can be activated by the presence of a reducing substance that reduces the lanthanide-doped material in a redox interaction. The lanthanide-doped material can be used for biodetection (e.g. detecting the presence of microorganisms in a sample). This could have numerous applications, such as detecting the presence of contaminating microorganisms in a sample (e.g. in a food or cosmetic product).

### BACKGROUND

Detecting bacterial contamination in food and other environments is commonly done using agar-plated Petri dishes to culture the bacteria. This technique is time-consuming and relies on labor-intensive procedures such as extraction, dilution, isolation, enrichment, counting, etc. Results may take 3-5 days for a bacterium determination or more than 7 days for yeasts and molds. To achieve faster results, a variety of different alternative techniques have been developed. One technique is a simplified version of the standard Petri dish, such as Petrifilm, Compact Dry, etc. This technique has been well-practiced throughout the world, but results still takes 2-4 days.

There are also rapid microbiology techniques that calculate the statistical most probable number (MPN) from data produced by miniaturized biochemical kits, antibody and nucleic acid-based assays, and modified conventional tests. Examples of products using such methods include Vidas (from BioMeriuex) and SimPlate (from BioControl). Growth-based techniques monitor microbial growth by measuring CO₂ levels (such as Neogen's Solaris/Biolumix system) or oxygen consumption (such as the Green Light system of Mocon/Luxcel Biosciences). These testing systems are generally more suitable for highly contaminated samples.

These techniques use automated detection with fast instrument calculations, replacing the need for visual counting of bacteria colonies and multiple dilution steps. However, these automated techniques also have their disadvantages. The antibody and/or enzyme reagents, such as those used in the Vidas and SimPlate products, have high cost. CO₂ detection methods that measure the indirect pH change caused by CO₂ generated from bacteria respiration in an air-tight environment has limitations when used with anaerobic microorganisms and colored samples. Oxygen consumption systems using a fluorescent green oxygen probe may be compromised by interference from biologic samples having intrinsic autofluorescence.

### SUMMARY

In one aspect, our invention is a particulate lanthanide-doped inorganic material. The material comprises an inorganic host phosphor that is capable of photoexcitation. As provided, the inorganic host phosphor is in an oxidized state such that its photoluminescence capability is suppressed. The inorganic host phosphor can be brought to this oxidized state by redox reaction with an oxidizing agent. In some embodiments, the lanthanide-doped inorganic material comprises an oxidizing agent. The particulate material further comprises a lanthanide ion dopant that is dispersed in the inorganic host phosphor and is capable of receiving the transfer of photoexcitation energy from the inorganic host phosphor. When the inorganic host phosphor is in a reduced state, energy transfer to the lanthanide ion dopant is allowed and after absorbing the energy, the lanthanide ion dopant emits light. In some embodiments, the inorganic host phosphor comprises cerium. In the “as provided” oxidized state, the cerium is oxidized cerium (IV), i.e. tetravalent cerium. When the cerium is reduced, the cerium is reduced cerium (III), i.e. trivalent cerium.

In another aspect, our invention is a method of biodetection. The method comprises contacting the sample with a particulate lanthanide-doped inorganic material of our invention. The lanthanide-doped inorganic material is then exposed to excitation light. Presence of biochemical substances in the sample that reduce the inorganic host phosphor may activate photoluminescence of the lanthanide-doped material. This luminescent light emitted from the lanthanide-doped inorganic material is detected. In some embodiments, this detection of the luminescent light is performed in conjunction with incubation of the sample.

In another aspect, our invention is a testing kit for biodetection. The biodetection kit comprises a particulate lanthanide-doped inorganic material and a sample container for holding the sample. At least a portion of the sample container is optically transparent. The particulate lanthanide-doped inorganic material could be provided separately or contained within the sample container. In some embodiments, the kit further comprises a liquid growth medium. The liquid growth medium could be provided separately or contained within the sample container. In some embodiments, the liquid growth medium is provided with the particulate lanthanide-doped inorganic material contained therein. In some embodiments, the sample container is provided with the growth medium and the particulate lanthanide-doped inorganic material contained therein (e.g. the particulate lanthanide-doped inorganic material is mixed in with the liquid growth medium).

In another aspect, our invention is a cell culture substrate onto which a sample may be applied for testing. The cell culture substrate comprises a planar foundation and a growth support coating on the planar foundation. The cell culture substrate further comprises a particulate lanthanide-doped inorganic material of our invention, which may be dispersed within the growth support coating, layered on the growth support coating, or both.

### BRIEF DESCRIPTION OF THE DRAWINGS

FIGS. 1-4 show an example of how our invention could operate for biodetection. FIG. 1 shows a conceptual view of the lanthanide-doped inorganic material. FIG. 2 shows photoexcitation in the host phosphor. FIG. 3 shows the lanthanide-doped inorganic material in the presence of a sample containing biologic material. FIG. 4 shows light emission from the lanthanide ion dopant.

FIG. 5 shows a sample vial containing a sample of biologic material and particles of the lanthanide-doped inorganic material.

FIG. 6 shows a schematic view of a fluorescence detection instrument for reading fluorescence from a sample.

FIG. 7 shows a cross-section side view of a cell culture substrate.

FIG. 8 shows the time profile of concentration dependence of E. coli at different inoculation levels (cfu/mL) in BPW (buffered peptone water).

FIG. 9 shows the time profile of concentration dependence of Pseudomonas spp. at different inoculation levels (cfu/mL) in TSB (tryptic soy broth).

FIG. 10 shows the time of profile of total bacteria in ground beef and beef chuck in BPW.

### DETAILED DESCRIPTION

Our invention relates to particulate lanthanide-doped inorganic materials, in which its photoluminescence capability is sensitive to the chemical environment. The lanthanide-doped inorganic material used in our invention is in particulate form, i.e. comprising particles. The individual particles may be in the nanometer or micrometer size ranges. In some embodiments, the average particle size in the particulate material is in the range of 1-999 nm in diameter; in some cases, in the range of 1-600 nm; in some cases, in the range of 1-500 nm; and in some cases, in the range of 50-400 nm. For the purpose of definition herein, the size of the particles is determined by survey view of a scanning electron micrograph. The distribution range of particle sizes may be wide or narrow depending on various factors, such as the method of fabrication, centrifuge, sieving or filtration technique applied, composition of the particles, etc. The particles may have any suitable shape. Having the material be made in particulate form can be beneficial for facilitating dispersability in liquids. Moreover, having the particles be sized sufficiently small (e.g. in the nanoscale range) could be beneficial in allowing the particles to enter inside the cells and detect the presence of intracellular metabolites (e.g. those reflecting the health of the cell).

The lanthanide-doped material comprises an inorganic host phosphor and a lanthanide ion dopant. The lanthanide-doped material is capable of photoluminescence (light emission after the absorption of photons), which is sensitive to the chemical composition of its surroundings. Because the photoluminescence activity (e.g. intensity, lifetime, etc.) depends on the surrounding chemical composition, the photoluminescence of the lanthanide-doped material can be used for detecting substances of interest in a sample.

Inorganic Host Phosphor.

Photoluminescence of the lanthanide-doped material relies on photoexcitation of the inorganic host phosphor and subsequent transfer of the photoexcitation energy to the lanthanide ion dopant. Any suitable inorganic phosphor can be used in our invention, including those that can produce fluorescent or phosphorescent light emission, and including down-converting phosphors which emit lower energy light than they absorb as well as up-converting phosphors which emit higher energy light than they absorb. Structurally, the host phosphor provides the bulk material into which the lanthanide ion dopants are dispersed. The host phosphor can have any suitable structural form, including being amorphous or crystalline (e.g. forming a crystal lattice into which the dopant ions are dispersed) or a combination of both.

The ability of the inorganic host phosphor to transfer its photoexcitation energy to the lanthanide ion dopant is dependent on the oxidation state of the host phosphor. The inorganic host phosphor is capable of being oxidized and reduced by a redox reaction with an oxidizing or reducing substance. As provided for use in biodetection, the host phosphor is in a relative oxidized state. Although the host phosphor is capable of photoexcitation, in this relative oxidized state, the photoexcitation capability is suppressed. But in the presence of a reducing substance, the host phosphor becomes reduced to a lower oxidation state. In this relatively lower oxidation state (reduced), photoluminescence of the lanthanide-doped inorganic materials is activated because the excitation energy of the host phosphor is transferred to the dopant ions (as will be explained below).

Among the lanthanides, cerium may be able to serve as the host phosphor. The reason for this is because unlike other lanthanides, cerium is capable of being oxidized from its Ce³⁺ trivalent form to a Ce⁴⁺ tetravalent form, and likewise, it can be reduced from the Ce⁴⁺ tetravalent form to its Ce³⁺ trivalent form. In some embodiments, the inorganic host phosphor is cerium phosphate. In some embodiments, the inorganic host phosphor comprises a lanthanide ion that is different from the lanthanide ion dopant.

Oxidizing Agent.

The inorganic host phosphor can be brought to its oxidized state by redox reaction with an oxidizing agent. In some embodiments, the composition includes one or more oxidizing agents that can oxidize the inorganic host phosphor in a redox interaction. Examples of oxidizing agents that could be used include: sodium percarbonate, hydrogen peroxide, hydrogen peroxide and transition metal mix, sodium perborate, sodium chlorite, sodium chlorate, sodium peroxydisulfate, calcium peroxide, hydrogen peroxide adducts such as poly(vinylpyrrolidone) hydrogen peroxide complex, urea hydrogen peroxide, urea hydrogen peroxide adduct, potassium permanganate, sodium dichromate, peroxymonosulfuric acid, sodium peroxydisulfate, sodium perchlorate, peroxydisulfuric acid, nitric acid, benzoyl peroxide, perchloric acid, ozone, sodium periodate, osmium tetroxide, peracetic acid, periodic acid, potassium peroxydisulfate, sodium bromate, sodium dichloroiodate, sodium hypochlorite, and lead (IV) acetate. Preferred are strong oxidizing agents such as sodium percarbonate, hydrogen peroxide, hydrogen peroxide and ferrous salt mix, sodium perborate, sodium chlorite, sodium chlorate, sodium peroxydisulfate, poly(vinylpyrrolidone) hydrogen peroxide complex, urea hydrogen peroxide, and calcium peroxide.

Lanthanide Ion Dopant.

One or more different types of lanthanide ions are dispersed in the composition as a dopant material. One or more different types of lanthanides may serve as the dopant in the composition. Among the lanthanides, samarium Sm(III), europium Eu(III), terbium Tb(III) and dysprosium Dy(III) ions are the most commonly used in fluorescence applications. In some embodiments, the lanthanide ion dopant is terbium(III) ion or europium(III) ion.

The lanthanide ion dopant serves as the emitter by absorbing the photoexcitation energy from the inorganic host phosphor. In particular, the inorganic host phosphor is excited by excitation light and this excitation energy is then transferred to the dopant ions. Absorbing this transferred energy, the dopant ions become excited and produce fluorescent or phosphorescent light emission.

In some embodiments, the molar amount of the lanthanide ion dopant in the composition is in the range of 0.5-30% that of the molar amount of the inorganic host phosphor. The optimal molar amount may be different for different lanthanides. Using dopant amounts in these ranges can avoid the problem of poor photonic performance from interactions between neighboring dopant ions, which can occur if the dopant amount is too high. When using terbium (III) ion as the dopant, in some cases, the molar amount ranges from 5-25% relative to the host substance, and preferably from 10-15%. When using europium (III) ion as the dopant, in some cases, the molar amount ranges from 0.5-10%, and preferably 1-5%.

The lanthanide-doped material can comprise various combinations of the inorganic host phosphor and lanthanide ion dopant. In some embodiments, the lanthanide-doped material is CeₓPO₄:Ln_{y} (x+y=1; Ln=elements Sm, Eu, Tb, or Dy). In some embodiments, the lanthanide-doped material is LaₓCe_{y}PO₄:Ln_{z} (x+y+z=1; Ln=elements Sm, Eu, Tb, or Dy). Preferably, the dopant amount y or z is 0.005-0.3 (0.5%-30%).

For CeₓPO₄:Tb_{y} (x+y=1), in some embodiments, the dopant amount y is in the range 0.05-0.2 (5-20%), and preferably, in the range 0.1-0.15 (10-15%). For CeₓPO₄:Eu_{y} (x+y=1), in some embodiments, the dopant amount y is in the range 0.005-0.1 (0.5-10%), and preferably, in the range 0.01-0.05 (1-5%).

Photoluminescence.

The luminescence emitted by the particulate lanthanide-doped inorganic material may be fluorescent or phosphorescent light emission. The terms “suppressed” and “activated” with respect to the photoluminescence of the lanthanide-doped material are not intended to indicate the absolute amounts of luminescence, but rather the relative intensity of the luminescence in comparison to each other. The intensity of the luminescence in the “suppressed” state is lower than the intensity of the luminescence in the “activated” state. Being in the “suppressed” state does not necessarily mean complete absence of luminescence. But in general, the operation of our invention is more effective when there is very low or non-detectable light emission in the “suppressed” state and much higher luminescence intensity in the “activated” state. This capability of the lanthanide-doped material to be converted from a “suppressed” state to an “activated” state of photoluminescence can sometimes be referred to as being “fluorogenic.”

It is not necessary that all the host phosphor be oxidized to result in suppression of photoluminescence in the lanthanide-doped material. It is possible that oxidation of only a small fraction of the host substance results in photoluminescence suppression. Likewise, it is not necessary that all the host phosphor be reduced to result in activation of photoluminescence in the lanthanide-doped material. It is possible that reduction of only a small fraction of the host phosphor results in photoluminescence activation.

In our invention, the sensitivity of the photoluminescence to the chemical environment arises from the fact that the photoluminescent operation depends on the oxidation state of the inorganic host phosphor. By exposing to an appropriate wavelength excitation light, the inorganic host phosphor is photoexcited. This photoexcitation energy is then transferred to the lanthanide ion dopant. This transferred energy is then emitted as light by the lanthanide ion dopant. This is the photoluminescent light emitted by the lanthanide-doped inorganic material.

However, whether the transfer of energy occurs between the excited host phosphor and the lanthanide ion dopant depends on the oxidation state of the host phosphor. In a relatively higher oxidation state, the transfer of energy from the host phosphor to the lanthanide ion dopant is blocked or substantially impeded. However, this energy transfer capability can be restored by reducing the oxidation state of the host phosphor. Switching the host phosphor to a relatively reduced state can occur by redox interaction with the chemical environment. Thus, when the lanthanide-doped inorganic material comes into contact with a redox reducing substance, the photoluminescence of the lanthanide-doped inorganic material may become activated.

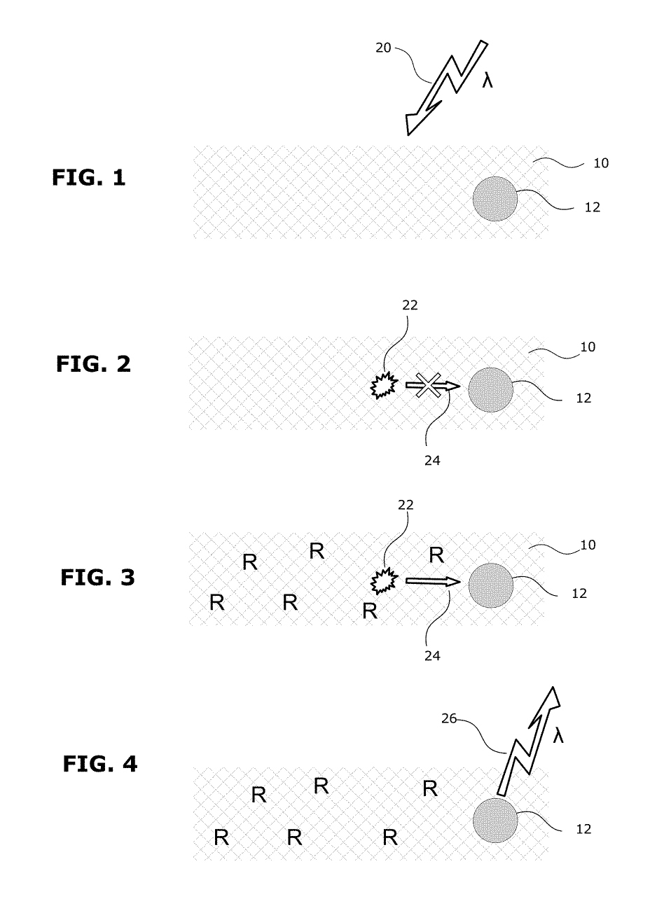

FIGS. 1-4 show an example of how our invention could operate for biodetection. FIG. 1 shows a conceptual view of the lanthanide-doped inorganic material, which comprises an inorganic host phosphor 10 that forms a crystalline matrix into which a lanthanide ion dopant 12 is dispersed. In operation, the lanthanide-doped inorganic material is exposed to excitation light 20. As shown in FIG. 2, this causes photoexcitation 22 in the host phosphor 10. This photoexcitation energy 22 would otherwise be transferred (shown by arrow 24) to the lanthanide ion dopant 12. However, because of the oxidized state of the host phosphor 10, this energy transfer does not occur and there is no luminescence from the lanthanide ion dopant 12 (and accordingly, no photoluminescence from the lanthanide-doped inorganic material). Thus, photoluminescence of the lanthanide-doped inorganic material is suppressed.

In FIG. 3, the lanthanide-doped inorganic material has been placed in a liquid containing a sample containing biologic material. The biologic material generates biochemical products (R) that are capable of reducing the host phosphor 10 by a redox interaction. As compared to FIG. 2, the photoexcitation 22 of the host phosphor 10 is transferred to the lanthanide ion dopant 12. As shown in FIG. 4, as a result, the lanthanide ion dopant 12 absorbs this energy and subsequently emits its own luminescence light 26.

Detection Method.

Because the lanthanide-doped material can detect the presence of reducing biochemical substances, it can be used for biodetection. As explained in more detail below, examples of biodetection include detecting the presence of microorganisms in a sample, quantifying the amount of microorganisms in a sample, testing for antibiotic susceptibility, screening for antibiotic drugs, and assessing for cell viability or function. As used herein, the term “detecting” in reference to luminescence or the content of the sample also encompasses measuring the luminescence or the content of the sample. The sample is contacted with the particulate lanthanide-doped inorganic material, in which its photoluminescence capability is suppressed. Contact with the sample may cause the inorganic host phosphor to become reduced (e.g. by redox reaction with biochemical substances in the sample). In the reduced state, photoluminescence of the particulate lanthanide-doped inorganic material is activated.

The lanthanide-doped material is then exposed to excitation light, which can have any suitable wavelength, depending upon the optical characteristics of the material. The excitation light may be provided over any suitable time frame, such as steady exposure (e.g. for detecting steady fluorescence) or pulsed excitation exposure (e.g. for time-delayed detection of fluorescence). In the activated state, the particulate lanthanide-doped inorganic material is able to emit light in response to the excitation light exposure. This luminescence may indicate that the biochemical substance of interest is present in the sample.

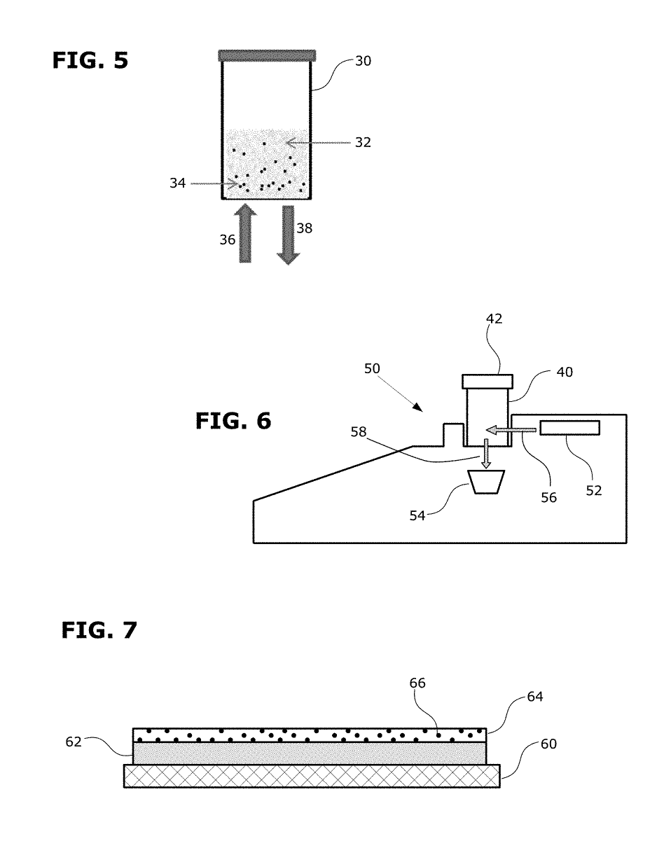

FIG. 5 shows an example of how this detection method could be applied to a sample of microorganisms contained in a sample vial 10. The sample vial 10 also contains a growth medium 32 for supporting the growth of the microorganisms. Nanoparticles 34 of the lanthanide-doped inorganic material are put into the sample vial 10 and dispersed therein. The microorganisms are incubated to allow growth and release of metabolic products into the sample material. One or more of the metabolic products act to reduce the lanthanide-doped nanoparticles 34 in a redox reaction. At the appropriate time, the sample is exposed to excitation light. Because photoluminescence of the lanthanide-doped inorganic material is now activated, the lanthanide-doped inorganic material emits photoluminescent light in response to the excitation light.

A single reading of the luminescence signal may be taken or multiple readings may be taken. Any suitable time parameter for reading the luminescence signals may be used. For example, the readings may be taken continuously, intermittently, or otherwise. In some embodiments, one or more detection readings are taken after a delay of time or multiple detection readings are taken over an interval of time (e.g. as in time-resolved fluorescence spectroscopy or fluorescence lifetime imaging). As used herein, the term “multiple” with respect to luminescence signal readings encompasses continuous readings over time, discrete readings over time, and otherwise.

Having one or more detection readings taken after a delay of time or multiple detection readings taken over an interval of time can be useful in various ways. For example, as explained below, taking the detection readings in this manner could be useful for samples containing cells to acquire data about the growth of the cells. In another example, if the sample contains a biologic material, the time parameters may be selected to avoid interference from the background autofluorescence that is present in many biologic samples. This can take advantage of the fact that lanthanide-doped inorganic materials can have relatively long luminescence lifetimes. Whereas a typical organic fluorescent dye (such as those present in biologic materials) has a luminescence lifetime within nanoseconds, a lanthanide-doped inorganic material could have a luminescence lifetime on the order of microseconds to milliseconds. Thus, by selecting the appropriate time parameters for luminescence detection, the background autofluorescence from a biologic sample could be avoided. In some embodiments, the method comprises taking a detection reading after a time delay of at least 100 nanoseconds after the exposure to excitation light is completed; in some cases, after at least 500 nanoseconds; and in some cases, after at least 1 microsecond.

Selecting the time parameters for luminescence detection could also be useful in situations where the luminescence lifetime of the lanthanide-doped material is sensitive to the chemical environment. For example, events such as rotational diffusion, resonance-energy transfer, and dynamic quenching can occur on the same time scale as fluorescence decay. Thus, the time parameters for luminescence detection could be selected to investigate these processes and gain insight into the chemical composition of the sample.

Useful Applications.

Because the photoluminescence of the lanthanide-doped inorganic material is sensitive to its chemical surroundings, it could be used to detect the presence of a reducing biochemical substance in a sample. The sample may come from any of various sources, including food products (including drinks, beverages, and food supplements), clinical specimens, rinses or swabs, water supply, environmental test sample, cosmetics, or from a sample collector such as a sponge, wipe cloth, cotton-tipped swabs (e.g. Q-tips), etc. The sample may be in any suitable physical form, including liquid or solid.

Besides the presence of the reducing biochemical substance itself, the presence of that biochemical may be indicative of other things, such as information about cells that are present in the sample. As used herein, the term “cell” means a biologic eukaryotic or a prokaryotic cell, including mammalian cells, microorganism cells, and plant cells. Microorganism includes yeasts, molds, and bacteria (including pathogenic, non-pathogenic bacteria, and background microflora). Cells have metabolic activity, and one or more products of their metabolism (e.g. by-products or waste products) may interact with the lanthanide-doped material in a redox reaction. Examples of such metabolic biochemicals that could be detected include NADH, sugars, hydrogen, reduced sulfur compounds, ethanol, acetate, lactate, and butyrate.

Thus, the luminescence detection readings could be used to identify the presence of cells (e.g. microorganisms) in a sample. Moreover, the luminescence detection readings could also be used to estimate the quantity of the cells in the sample. The term “quantity” with respect to measuring the cells encompasses any of the various types of measures for determining the number of cells, including absolute number, density (such as cfu/mL), concentration, weight, functional units, etc.).

For samples containing cells, growth media may be added to the sample. The growth medium and culture conditions can be selected according to the circumstances. For example, to obtain total viable counts (TVC), a growth medium that supports the growth of all microorganisms that could be present in a sample may be used. In another example, alternatively, a growth medium that supports the growth of a specific group or strain of microorganism may be selected. Examples of growth media that can be used include buffered peptone water, tryptic soy broth, and Plate Count Broth (Difco, Becton Dickinson). There are also various types of selective growth media suitable for the growth of yeasts and molds, or selective groups of microorganisms such as E. coli, Staphylcococcus, Pseudomonas, Salmonella and Listeria, or lactic acid-producing bacteria.

Culture conditions may be selected to optimally grow a specific type of microorganism of interest. For example, growth of E. coli may be optimally supported at a temperature around 37° C., while growth of yeast or mold may be optimally supported at a lower temperature, such as 24° C. In another example, growth of aerobic microorganism could be supported by selecting appropriate aerobic conditions and growth of anaerobic microorganisms could be supported by selecting an anaerobic condition, such as a CO₂ atmosphere.

The sample may require an incubation time to allow the microorganisms to grow and multiply. As the microorganisms (if any present) multiply and release their metabolic products, more of the lanthanide-doped material is activated to luminesce. The increased photoluminescence output of the lanthanide-doped material is detected as described above.

As explained above, selecting the time parameters for luminescence detection could also be useful in various situations. With respect to samples containing cells, taking one or more detection readings after a delay of time or multiple detection readings over an interval of time can provide useful information. For example, in a sample containing cells, tracking the photoluminescence output over time may provide useful data about the growth pattern of the microorganism, rate of growth, initial number of microorganisms, etc. For example, a plot of the photoluminescence output vs. time may resemble the pattern of microorganisms going through a lag phase, exponential phase, and final static phase of growth. As such, the initial photoluminescence activation time may correlate with the initial number of microorganisms present in a sample as they transition from lag phase to exponential phase of growth. An earlier activation time would correlate with a greater the number of microorganism present in the sample. This activation time may depend on the type of microorganism, growth media, or temperature. By fixing controllable growth conditions (such as incubation temperature and optimized growth medium), a particular type of microorganism can exhibit reproducible activation times that are proportional to the initial numbers of microorganism in a sample.

Selection of the time parameters for luminescence detection could be made to correspond with time periods for incubation of cell-containing samples. In some embodiments, the multiple detection readings are taken over a time interval that is up to 72 hours after beginning incubation of the sample; in some cases, within 2-72 hours; in some cases, within 2-24 hours; and in some cases, within 2-12 hours. The multiple detection readings over these time periods may be taken intermittently. For example, the readings could be taken at time points separated by one or more intervals (not necessarily identical) that are in the range of 5 minutes to 1 hour (e.g. every 15 minutes or every 30 minutes). Incubation of the sample may be performed at any suitable temperature to grow the cells. In some embodiments, the sample is incubated at a temperature that is above 20° C.

Detection of reducing biochemical substances could be applied in a wide variety of different contexts, such as detecting the presence of microorganisms, estimating the amount of microorganisms, identifying the type of microorganism, monitoring the metabolic health of cells. The ability to detect microorganisms can have a number of useful applications. One example is for detecting microorganism contamination of food products. In this situation, the sample comprises a food product.

Our biodetection method could also quantify the amount of cells present in the sample. The culture conditions can be selected such that incubation of the sample will result in generating a signal that is proportional to the number of viable cells that are present in the sample. In this situation, the sample would comprise cells (e.g. such as microorganisms).

Our biodetection method could also be used for testing the susceptibility of microorganisms to antibiotic drugs. For example, microorganisms present in a clinical specimen could be incubated in a growth medium containing an antibiotic drug of interest along with the particulate lanthanide-doped material of our invention. The concentration of the antibiotic drug may be varied to determine the minimum effective concentration for inhibiting the growth of the microorganism (sometimes referred to as the minimum inhibition concentration or MIC). The lack of photoluminescence activation (as compared to a control sample without the antibiotic drug) could indicate that the growth of the microorganism is being inhibited by the antibiotic drug. In this situation, the sample would comprise a microorganism and an antibiotic drug.

Our biodetection method could also be used for screening antibiotic drugs during the drug development process, including primary screening or secondary screening of new antibiotic drugs. For example, high throughput screening is widely used in the pharmaceutical industry for screening a drug library to find drug candidates that inhibit the growth of particular microorganisms. Microorganisms of interest could be incubated in a growth medium containing a candidate antibiotic drug along with the particulate lanthanide-doped material of our invention. If there is a lack of photoluminescence activation (as compared to a control sample without the antibiotic drug), this could indicate that the growth of the microorganism is being inhibited by the candidate antibiotic drug. This candidate drug could be selected as a “lead” drug candidate for further preclinical development. In this situation, the sample would comprise a microorganism and an antibiotic drug.

Our biodetection method could also be used for confirming sterility (or detecting contamination) in medical products such as medical devices or drugs. For example, the particulate lanthanide-doped material of our invention could be added to a pharmaceutical rinse sample or a medical device rinse sample containing a non-selective growth medium which supports all microorganisms in the sample. After incubating the sample, the photoluminescence signal could be read to determine the presence or absence of microorganisms and the total viable count. A positive photoluminescence signal that exhibits a phase of exponential increase could indicate that the sample is contaminated. In this situation, the sample would comprise a rinse sample.

The biodetection method of our invention could also be used for cell viability or function assays. In this situation, the sample would comprise cells. When cells die, they rapidly lose the ability to convert the substrate to product. Thus, measuring the metabolic product(s) of cells can serve as a marker of cell viability, cell proliferation and many important live-cell functions, including apoptosis, cell adhesion, chemotaxis, multidrug resistance, endocytosis, secretion, and signal transduction. One or more such metabolic products can be measured by our biodetection method to assay for cell viability or function. Because the lanthanide-doped material can be non-toxic or have relatively low toxicity (e.g. as in the case of CePO₄:Tb or CePO₄:Eu), this material can be useful for cell viability or function assays.

As demonstrated here, our invention has numerous applications. Examples of microorganisms that could be detected by our invention include bacteria (such as E. coli, Salmonella, Campylobacter, Listeria, Clostridium, Pseudomonas, Staphylococcus, Streptococcus, Mycoplasma, etc.) and fungus (such as Aspergillus, Candida, mold, etc.). Our invention could be used to detect such microorganisms in a variety of different scenarios, including food safety, water safety, cosmetics safety, drug screening, clinical diagnostics, cell viability assays, industrial microbiology (e.g. in the production of chemicals through fermentation), nutraceutical safety, animal feed contamination, pharmaceutical contamination, environmental safety assessment, and quarantine control (e.g. for customs screening).

Testing Kit.

Any suitable instrument could be used for photoluminescence detection. Examples of such instruments include fluorescence spectrometers, fluorescence imaging systems, microtiter plate readers, fluorescence microscopes, microplate readers, flow cytometers, etc. Examples of such instruments include 96-well microtiter plate fluorescence readers manufactured by BioTek, PerkinElmer, TECAN, Molecular Devices, etc. Our invention also encompasses a kit for biodetection that can be used in conjunction with such photoluminescence detection instruments. The biodetection kit comprises the particulate lanthanide-doped inorganic material of our invention.

The biodetection kit furthers comprises a sample container for holding the sample to be tested. The sample container may be any type of container suitable for holding a liquid material to be analyzed for photoluminescence. Examples of sample containers that could be used include vials, bottles, jars, or dishes. The sample container can be designed to hold a single sample or multiple samples (e.g. a multiwell plate). The sample container may hold any suitable volume of liquid. For example, the sample container may hold a volume in the range of 0.5-15 mL. Examples of such volumes that could be used include about 0.5, about 1, about 1.5, about 2, about 3, about 10, and about 15 mL. For sample containers that are designed to hold multiple samples, the volume of each well may be in the range of 5-300 μL. Example well volumes that could be used include about 5, about 10, about 50, about 100, about 150, about 200, about 250, and about 300 μL.

At least a portion of the sample container is optically transparent (e.g. made with transparent glass or plastic) to allow for transmission of excitation or photoluminescent light. For example, the entire sample container may be made of transparent glass. In another example, the sample container may have one or more transparent windows (e.g. on the side or bottom of the container). In some cases, the sample container is a closed container with or without an air permeable cap. The sample container may be single use, disposable, reusable, etc.

In our kit, the sample container may be provided with the particulate lanthanide-doped inorganic material contained therein; or the particulate lanthanide-doped inorganic material may be provided separately (e.g. the user pours the material into the sample container at the time of use). The biodetection kit may further comprise a liquid growth medium. The sample container may be provided with the growth medium contained therein; or the growth medium may be provided separately (e.g. the user pours the growth medium into the sample container at the time of use). As explained above, the growth medium can be selected from among any of the various types of growth media that are available.

In some embodiments, the growth medium contains the particulate lanthanide-doped inorganic material of our invention (e.g. lanthanide-doped nanoparticles may be premixed with the growth medium); or the particulate lanthanide-doped inorganic material may be provided separately from the growth medium (e.g. the user pours the material into the growth medium at the time of use). The growth media can be in any suitable form, including dehydrated, semi-solid, or liquid form.

FIG. 6 shows an example of how a biodetection kit of our invention could be used. The kit includes a sample bottle 40 which, as provided, contains a growth medium and the particulate lanthanide-doped inorganic material premixed therein. At the time of use, the user opens the cap 42 on the sample bottle 40 and places the sample therein. After mixing to ensure adequate dispersion of the particulate material, the sample is incubated at the appropriate temperature. After incubation, the sample bottle 40 is placed onto a fluorescence detection instrument 50. The excitation light source 52 of the instrument 50 is turned on to expose the sample to excitation light 56. The photodetector 54 of the instrument detects fluorescence 58 being emitted from the particulate material.

Another embodiment of our invention is a cell culture substrate onto which a sample may be applied for testing. The cell culture substrate comprises a planar foundation (e.g. paper, film, sheet, dish, etc.) and a growth support coating on the planar foundation. The planar foundation may have any suitable degree of flexibility or stiffness, and be made of any suitable material such as paper or plastic. The growth support coating comprises materials that promotes the growth of cells (such as microorganisms) or provides structural support for the growth of cells. For example, the growth support coating may comprise nutrients, dehydrated growth media, adhesives, or gelling agents (e.g. hydrogels). The growth support coating may be a single layer or made up of multiple separate layers. Examples of planar foundations and growth support coatings that can be used in our invention are described in U.S. Pat. No. 4,565,783; No. 5,364,766; and No. 8,846,335, which are incorporated by reference herein.

The cell culture substrate further comprises a particulate lanthanide-doped inorganic material of our invention, which may be dispersed within the growth support coating, layered on the growth support coating, or both. FIG. 7 shows an example of a cell culture substrate of our invention. The cell culture substrate comprises a sheet 60 for its base. On this sheet 60, there is a growth support coating, which comprises a layer of dehydrated growth medium 62 and a thin layer of gelling agent 64. Nanoparticles 66 of the lanthanide-doped inorganic material are dispersed within the gelling agent layer 64. This cell culture substrate can be used by applying a liquid sample of microorganisms onto the gelling agent layer 64 and then incubating for the growth of the microorganisms. With photoluminescence activation of the nanoparticles 66, the colonies of the microorganisms can be visualized by exposure to excitation light and be counted. The luminescence could also be detected using a luminescence detection instrument as described above.

### EXPERIMENTAL EXAMPLES

One example of a lanthanide-doped inorganic material that can be used in our invention is cerium phosphate doped with trivalent terbium ions (CePO₄:Tb³⁺). In this material, Tb³⁺ is excited by energy transfer from light-absorbing Ce³⁺. When the cerium ion is in a trivalent state, this Ce³⁺→Tb³⁺ energy transfer occurs. However, if the cerium ion is oxidized to its tetravalent state Ce⁴⁺, this energy transfer no longer occurs. Thus, suppression and activation of photoluminescence can be controlled by a Ce³⁺/Ce⁴⁺ redox reaction.

### Example 1—Preparing Fluorogenic CePO₄:Tb Nanoparticles Containing 15% (Molar) of Tb(III)

3.689 grams of Ce(NO₃)₄.6H₂O (Sigma-Aldrich) and 0.68 gram of Tb(NO₃).6H₂O (Sigma-Aldrich) were dissolved in 250 mL of deionized water under constant stirring at room temperature in a 1000 mL beaker. In another beaker, 2.4 grams of sodium phosphate monobasic monohydrate (Sigma-Aldrich) was dissolved in 250 mL water. To the first solution, the sodium phosphate monobasic solution was slowly added in a dropwise manner under constant stirring for overnight duration. The reaction was split into half, each having about 250 mL of volume. To the first beaker, 10 grams of sodium percarbonate was added. To the second beaker, 5 grams of sodium perborate was added. The reactions were allowed to continue for 4 hours at room temperature. The resulting solutions were aliquot into 50 mL conical centrifuge tubes and centrifuged at 3000 rpms for 15 minutes. The supernatant in each tube was decanted and the resulted precipitates were washed with 45 mL water three times. The tubes containing sodium carbonate were then merged into one tube using ethanol as solution. This same procedure was applied to the sodium perborate samples. The samples were then centrifuged again at 3000 rpm for 15 minutes. The resulting supernatant was decanted and the solid material at the bottom of the tube was dried at 60° C. for overnight duration.

### Example 2—Preparing Fluorogenic CePO₄:Tb Nanoparticles Containing 10% (Molar) of Tb(III)

2.46 grams of Ce(NO₃)₄.6H₂O (Sigma-Aldrich) and 0.374 gram of Tb(NO₃).6H₂O (Sigma-Aldrich) were dissolved in 200 mL of deionized water under constant stirring at room temperature in a 1000 mL beaker. In another beaker, 1.18 grams of potassium phosphate monobasic monohydrate (Sigma-Aldrich) was dissolved in 200 mL water. To the first solution, the sodium phosphate monobasic solution was slowly added in a dropwise manner under constant stirring for overnight duration. To the reaction, 20 grams of sodium percarbonate was added and the reaction was allowed to continue for 4 hours at room temperature. The resulting solutions were aliquot into 50 mL of conical centrifuge tubes and centrifuged at 3000 rpms for 15 minutes. The supernatant in each tube was decanted and the resulting precipitates were washed with 45 mL water three times. The tubes were then merged into one tube using ethanol as solution and again centrifuged at 3000 rpm for 15 minutes. The resulting supernatant was decanted and the solid material at the bottom of the tube was dried at 60° C. for overnight duration.

### Example 3—E. coli Culture

A 96-well, black rimmed Costar plate (Corning) was aseptically coated with 25 μl of a 2 mg/ml fluorogenic nanoparticle suspension of Example 1 in isopropanol. Handling the nanoparticles in isopropanol suspension facilitated more accurate dispensing of the same amount of nanoparticles in each well. The isopropanol was evaporated off the plate by leaving it to dry overnight. A fresh culture of E. coli (ATCC #51813) was incubated overnight in buffered peptone water (BPW). The bacterial culture was diluted 10-fold by mixing 1 mL of the original sample into 9 mL of BPW solution. This 10-fold dilution was repeated in series from each preceding dilution nine times, resulting in a series of 10-fold dilution nine times.

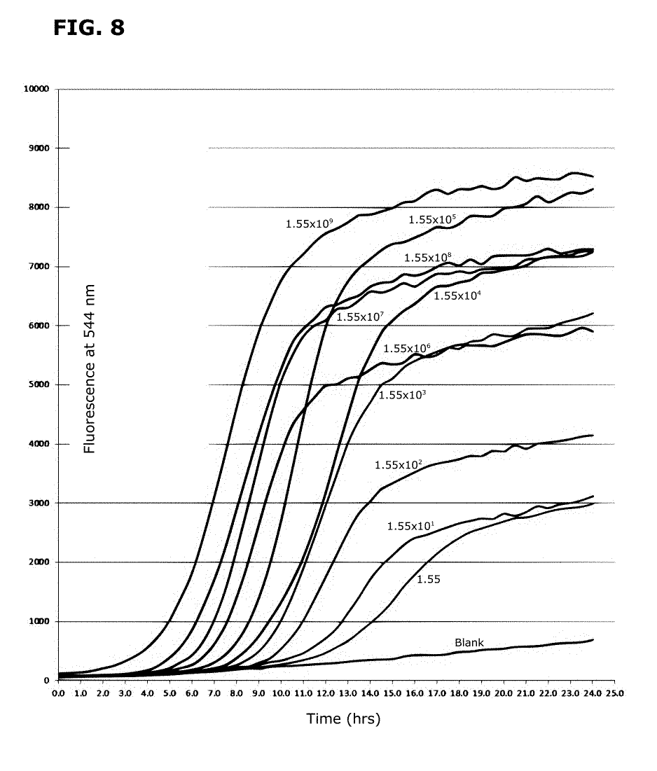

250 μl of each diluted sample is used to inoculate each well in duplicates. The 96-well plate was incubated at 37° C. and measured in a BioTek Synergy H1 microtiter plate reader with excitation light set at 300 nm wavelength and emission detection set at 544 nm wavelength at 30 minutes intervals. For comparison with the traditional Petrifilm count technique, two AC Petrifilms were also inoculated with the seventh dilution (10²) to get an estimation of the total viable counts (TVC) in the samples. The Petrifilms were then incubated at 37° C. for 24 hours before being counted. The counts obtained from AC plate was used to back-calculate the E. Coli concentration in each diluted sample. The results are shown in FIG. 8, which shows a plot of the measured fluorescence intensity over time for each of the diluted samples.

### Example 4—Pseudomonas Culture

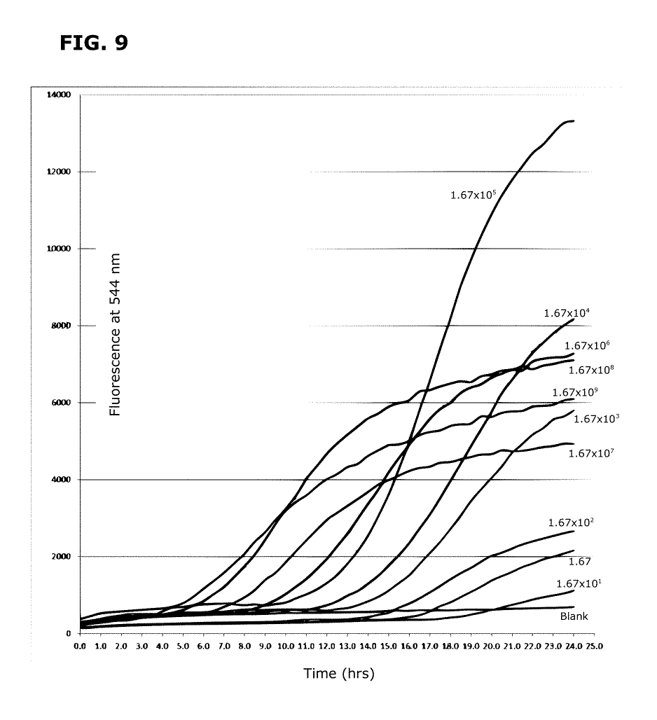

A 96-well, black rimmed Costar plate (Corning) was coated with fluorogenic nanoparticles in the same manner as above for Example 3. A fresh culture of Pseudomonas species (ATCC #51821) was incubated at 30° C. in trypticase soy broth (TSB) for 32 hours. The bacterial culture was serially diluted in TSB nine times in the same manner as described above for Example 3. 250 μl of each diluted sample is used to inoculate each well in duplicates. The plate was incubated at 30° C. and then read in the same manner as described above for Example 3. For comparison with the traditional Petrifilm count technique, two AC Petrifilms was inoculated with bacteria in the same manner as described above for Example 3. The Petrifilms were then incubated at 30° C. for 48 hours before counted. The counts obtained from AC plate was used to back-calculate the Pseudomonas species concentration in each diluted sample. The results are shown in FIG. 9, which shows a plot of the measured fluorescence intensity over time for each of the diluted samples.

### Example 5—Testing on Ground Beef and Beef Chuck Meat

A 96-well, black rimmed Costar plate (Corning) was coated with fluorogenic nanoparticles in the same manner as above for Example 3. Ground beef and beef chuck were purchased from a local supermarket to use as experimental samples. Following the standard microbiological technique for meat testing, 10 grams of the beef product was dropped into 90 mL of BPW in a sterile 7×12″ polyethylene bag and blended for 1 minute in a Stomacher 400 (A. J. Seward) at 250 rpm for 2 min.

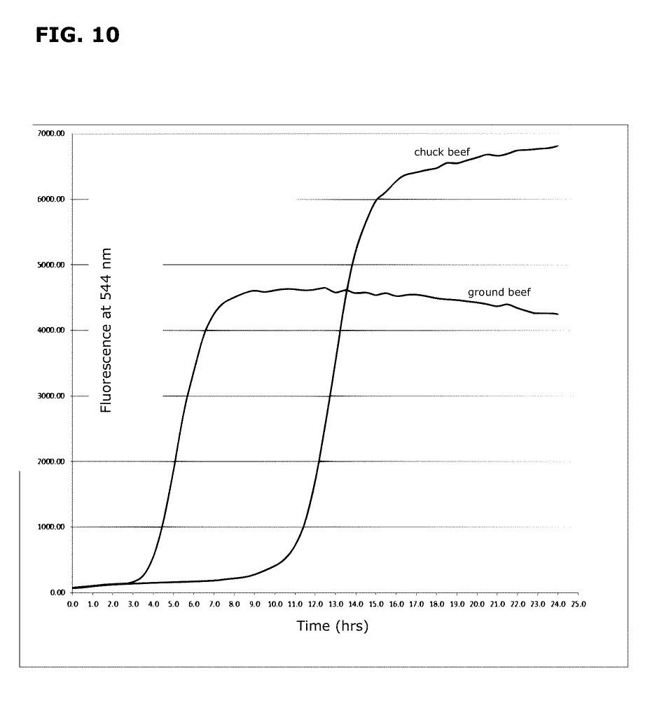

Each well was inoculated with 250 μl of a diluted sample. The 96-well plate was incubated at 30° C. and fluorescence measured in a BioTek Synergy H1 microtiter plate reader with excitation at 300 nm and emission detection at 544 nm read at 30 minutes intervals. The sample was serially diluted 10 times in BPW. Each dilution had two replicates for the inoculated AC Petrifilm plates. The samples were incubated at 30° C. for at least 24 hours before calculating the total viable counts (TVC) of bacteria. The results are shown in FIG. 10, which shows a plot of the measured fluorescence intensity over time.

The AC Petrifilm plate revealed that the beef chuck sample had about 1×10³ cfu/gram of meat while the ground beef sample had about 4.3×10⁶ cfu/gram. With regards to the fluorescence signals, the ground beef sample (determined to have 1×10³ cfu/gram bacteria load) showed initial “turn-on” time at around 3.5 hours; whereas the beef chuck sample (determined to have 4.3×10⁶ cfu/gram bacteria load) showed initial “turn-on” time at around 10.5 hours. The earlier turn-on time for the ground beef sample (compared to the beef chuck sample) correlates with its higher load of bacterial contamination.

In an alternate embodiment, instead of the inorganic host being a phosphor material, the inorganic host may be an electroluminescent material in which light emission is driven by electrical current. The foregoing description and examples have been set forth merely to illustrate our invention and are not intended to be limiting. Each of the disclosed aspects and embodiments of our invention may be considered individually or in combination with other aspects, embodiments, and variations of our invention. In addition, unless otherwise specified, the steps of the methods of our invention are not confined to any particular order of performance. Modifications of the disclosed embodiments incorporating the spirit and substance of our invention may occur to persons skilled in the art, and such modifications are within the scope of our invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A biodetection kit for testing a sample comprising:
a particulate lanthanide-doped inorganic material comprising:
an inorganic host phosphor in a first oxidation state and capable of being reduced to a second oxidation state, the second oxidation state being a relatively lower oxidation state than the first oxidation state; and
a lanthanide ion dopant dispersed in the inorganic host phosphor;
a sample container for holding the sample, at least a portion of the sample container being optically transparent.

2. The biodetection kit of claim 1, wherein the sample container is provided with the particulate lanthanide-doped inorganic material contained therein.

3. The biodetection kit of claim 1, further comprising a liquid growth medium.

4. The biodetection kit of claim 2, wherein the sample container is provided with the growth medium contained therein.

5. The biodetection kit of claim 2, wherein the liquid growth medium is provided with the particulate lanthanide-doped inorganic material contained therein.

6. The biodetection kit of claim 2, wherein the sample container is provided with the growth medium and the particulate lanthanide-doped inorganic material contained therein.

7. The biodetection kit of claim 1, wherein the lanthanide ion dopant is terbium(III) ion or europium (III) ion.

8. The biodetection kit of claim 1, wherein the molar amount of the lanthanide ion dopant is in the range of 0.5-30% that of the molar amount of the inorganic host phosphor.

9. The biodetection kit of claim 1, wherein the average size of the particles in the particulate lanthanide-doped inorganic material is in the range of 1-600 nm.

10. The biodetection kit of claim 1, wherein the lanthanide ion dopant is a first lanthanide ion and the inorganic host phosphor is a second lanthanide ion that is different from the first lanthanide ion.

11. The biodetection kit of claim 10, wherein the inorganic host phosphor comprises a cerium ion.

12. The biodetection kit of claim 1, wherein the particulate lanthanide-doped inorganic material further comprises an oxidizing agent that reacts with the inorganic host phosphor to maintain the inorganic host phosphor in the first oxidation state.

13. A method of biodetection in a sample, comprising:
contacting the sample with a particulate lanthanide-doped inorganic material of claim 1;
exposing the lanthanide-doped inorganic material to excitation light; and
detecting for emission of luminescent light from the lanthanide-doped inorganic material.

14. The method of claim 13, wherein the sample comprises a growth medium.

15. The method of claim 13, further comprising adding a growth medium to the sample.

16. The method of claim 13, wherein the step of detecting for light emission comprises making a detection reading after a delay of time from when the exposure of the lanthanide-doped inorganic material to excitation light is completed.

17. The method of claim 16, wherein the delay of time is at least 100 nanoseconds after the time when exposure to excitation light is completed.

18. The method of claim 13, wherein the step of detecting for light emission comprises making multiple detection readings over an interval of time.

19. The method of claim 18, further comprising incubating the sample, and wherein the interval of time is in a range from 2-72 hours after beginning the incubation.

20. The method of claim 13, wherein the method is for detecting a cell in a sample, and wherein the sample comprises a growth medium for promoting the growth of the cells.
